(19) United States
(12) Patent Application Publication
(10) Pub. No.: US 2004/0250314 A1
(43) Pub. Date: Dec. 9, 2004

(54) Transformed Brassica CC genome comprising Brassica AA transparent seed coat gene

(76) Inventors: Muhammed Habibur Rahman, Maribo (DK); Morten Jorsboe, Nykobing F (DK); Morten Helt Poulsen, Holeby (DK)

Correspondence Address: FROMMER LAWRENCE & HAUG, 745 FIFTH AVENUE- 10TH FL., NEW YORK, NY, 10151, US

(21) Appl. No.: 10/876,889

(22) Filed: Jun. 25, 2004

Related U.S. Application Data
(63) Continuation of application No. 09/489,817, filed on Jan. 24, 2000.

(30) Foreign Application Priority Data
Jan. 27, 1999 (GB) 9901814.5

(51) Int. Cl. C12N 15/82; C12N 15/87; A01H 5/00; C12N 5/10; A01H 5/10

(52) U.S. Cl. 800/281; 435/468; 435/419; 800/298; 800/278

(57) ABSTRACT

A transformed plant is described. The transformed plant comprises an exogenous transparent seed coat gene obtained from an AA genome.

The present invention relates to an organism. In particular, the present invention relates to a transformed plant, as well as tissues or cells thereof, and to transformed cells or tissues.

The Brassica napus (AACC, 2n=38) species is an amphidiploid species which originated in ancient times from the diploid species B. campestris (AA, 2n=20) and B. oleracea (CC, 2n=18) through interspecific hybridization. All of the naturally occurring strains of B. napus (which include oilseed rape) are of the black-seeded type.

In the Brassica species, the yellow seed is superior over the brown/black seed because of its higher oil and protein content, its lower fibre content and its aesthetic appearance in seed meal. For example, the yellow or partly yellow seed from B. campestris is associated with a 2-5% higher oil content, a 1% higher protein content and a 4-7% lower fibre content (Stringam et al. 1974; Daun and DeClercq 1988; Hu 1988; Simbaya et al. 1995); the yellow seed from B. juncea is associated with a 2.8% higher oil content, a 3.5% higher protein content and a 7.3% lower fibre content (Woods 1980; Simbaya et al. 1995); the yellow seed from B. carinata is associated with a 3.8% higher protein content and a 5.7% lower fibre content (Simbaya et al. 1995); and the seeds from the brown or yellow-brown seeded B. napus developed by cross breeding contain a 2-4% higher oil and protein content and a 3-7% lower fibre content (Shirzadegan and Röbbelen 1985; Hu 1988; Simbaya et al. 1995).

As most of the seed oil and protein are localised in the embryo, the seed coat contains very small amounts of oil and protein. Furthermore, in yellow seed, the proportion of seed coat to whole seed is 15% lower than that of the brown seed turnip rape (B. campestris) (Stringam et al. 1974). Thus, the higher oil and protein content in yellow seed is predominantly due to a larger proportion of embryo compared to the whole seed. The lower fibre content in meal derived from yellow or light coloured seed is largely due to a lower proportion of coat when compared to the whole seed (Stringam et al. 1974; Anjou et al. 1977). The seed coat pigment of Brassica is composed mainly of polyphenols which are polymers of leucocyanidins (Leung et al. 1979; Hu 1988). The polyphenol is deposited in the palisade and crushed parenchyma layers of the seed coat (Vaugan 1970; Stringam et al. 1974). The palisade layer is the prominent cell layer of the seed coat and is assumed to yield a higher fibre content and a lower oil and protein content. This palisade cell layer is reduced from ½ to ⅔rd in the yellow seed of turnip rape (Stringam et al. 1974). Consequently, lower proportions of polyphenol and lignin are expected from the yellow seed (Anjou et al. 1977; Theander et al. 1977; Slominski et al. 1994). As a result, the digestibility of seed coat or meal from yellow seed is significantly higher than that from black seed (Bell and Shires 1982; Slominski et al. 1994).

In view of the aforementioned advantages associated with yellow seed, it would be desirable to produce transformed cells capable of yielding yellow colour seeds and/or yellow-seeded plants.

According to a first aspect of the present invention there is provided a transformed CC genome comprising an exogenous transparent seed coat gene obtained from an AA genome. The transparent seed coat gene provides the yellow colour seed, as the coat is transparent thus enabling the yellow interior of the seed to be visualised. The visible yellow seed is produced by a substantially transparent seed coat which thereby exposes the natural embryo to the naked eye.

As used herein, the term “exogenous transparent seed coat gene” means a transparent seed coat gene having a different origin to other genes found on a particular genome.

The CC genome is found in some Brassica. Hence, the CC genome is obtainable from Brassica. Typically the CC genome is obtained from Brassica. Alternatively expressed the CC genome is a Brassica CC genome.

The AA genome is found in some Brassica. Hence, the AA genome is obtainable from Brassica. Typically the AA genome is obtained from Brassica. Alternatively expressed the AA genome is a Brassica AA genome.

Preferably the AA genome is obtained from any one of Brassica campestris, Brassica napus and Brassica juncea.

Preferably the AA genome is obtained from Brassica campestris.

Alternatively expressed, the present invention provides a transformed Brassica CC genome, wherein the transformed Brassica CC genome comprises a transparent seed coat gene obtained from a Brassica AA genome.

Preferably the CC genome is a transformed Brassica napus genome.

The visible yellow seed is produced by a substantially transparent seed coat which thereby exposes the natural embryo to the naked eye. This is in direct contrast to the black seeded and yellow-brown seeded Brassica plant seeds which have darker colours due to dark colouration and dark pigmentation in the seed coat.

According to a second aspect of the present invention there is provided a transformed plant, plant cell or plant tissue comprising an exogenous transparent seed coat gene.

In accordance with the present invention, the term “transparent seed coat gene”—which may be interchanged with the term “transparent seed coat colour gene” or the term “yellow seed coat gene” or the term “transparent yellow seed coat gene”—includes one or more nucleotide sequences, but preferably at least two nucleotide sequences which are capable of imparting a transparent seed coat. If there are at least two nucleotide sequences, then they may be located on the same chromosomal locus or on different chromosomal loci.

Thus, the term “transparent seed coat gene” of the present invention can be alternatively stated as being a gene or. a number of genes capable of making a seed coat substantially transparent and thereby enabling visualisation of the substantially natural yellow colour of the inner embryo component of the seed.

A seed coat is substantially transparent when there is a substantial absence of seed coat pigmentation when compared with the wild type seed coat.

As used herein, the term “wild type” means a form of a gene of allele that is considered the “standard” or most common type found in nature.

According to a third aspect of the present invention there is provided a transformed plant, plant cell or plant tissue comprising an exogenous transparent seed coat gene obtained from an AA genome.

Preferably the transformed plant, plant cell or plant tissue comprising an exogenous transparent seed coat gene is obtained from the AA genome from any one of Brassica campestris, Brassica napus and Brassica juncea.

Preferably the transformed plant, plant cell or plant tissue comprising an exogenous transparent seed coat gene is obtained from the AA genome of Brassica campestris.

Preferably, the transformed plant, plant cell or plant tissue is a transformed Brassica plant, plant cell or plant tissue.

Preferably, the transformed plant, plant cell or plant tissue is a transformed Brassica napus plant, plant cell or plant tissue.

In some embodiments, preferably the transformed Brassica plant is non-sterile (ie fertile).

Preferably the transformed Brassica plant, plant cell or plant tissue is capable of yielding seeds with a transparent seed coat or is capable of yielding plants having seeds with a transparent seed coat.

According to a fourth aspect of the present invention there is provided a method for increasing the levels of seed oil and protein and reducing the levels of fibre in a seed wherein the method comprises transferring the transparent seed coat gene of an AA genome of a first Brassica plant, tissue or cell into a CC genome of a second Brassica plant, plant tissue or plant cell.

The method of the present invention may further comprise the optional steps of: (i) selecting yellow seeds from black seeds or brown seeds; and/or (ii) comparing levels of erucic acid and glucosinolate(s) between the yellow seeds and the black seeds and the brown seeds.

Preferably the method comprises transferring the transparent seed coat gene of an AA genome of a first Brassica plant, plant tissue or plant cell into a CC genome of a second Brassica plant, plant tissue or plant cell.

Preferably the method comprises transferring the transparent seed coat gene of an AA genome of a first Brassica plant, plant tissue or plant cell into a CC genome of a second Brassica plant, plant tissue or plant cell.

According to a fifth aspect of the present invention there is provided a transformed Brassica napus plant capable of yielding seeds with a transparent seed coat.

According to a sixth aspect of the present invention there is provided a seed oil or a seed meal comprising an oil and protein content of at least about 70% seed dry matter and a fibre content of not more than about 8% oil free meal.

Preferably the seed oil or a seed meal comprises an oil and protein content of from about 70% to about 80% seed dry matter.

Preferably the seed oil or a seed meal comprises a fibre content of not more than about 6% to about 8% oil free meal.

According to a seventh aspect of the present invention there is provided a use of an AA genome as a vector for delivery of one or more genes of interest to an heterologous genome.

According to a eighth aspect of the present invention there is provided a transparent seed coat encoded by a transparent seed coat gene obtainable from NCIMB 40991 and/or NCIMB 40992.

According to a ninth aspect of the present invention there is provided a transparent seed coat.

Other aspects of the present invention are presented in the accompanying claims and in the following description and drawings.

Preferably any one or more of the transformed plant, the transformed cell or the transformed tissue is prepared by use of the embryo rescue technique.

Preferably any one or more of the transformed plant, the transformed cell or the transformed tissue is prepared by a process which comprises a chromosome doubling step using an agent that causes chromosome doubling - such as colchicine.

Preferably any one or more of the transformed plant, the transformed cell or the transformed tissue is prepared by a process which comprises an embryo-rescue step.

The present invention is advantageous in that it is now possible to produce yellow-seeded Brassica plants. The present invention is further advantageous in that it is now possible to produce yellow-seeded Brassica napus plants.

Thus, in accordance with the present invention, we have been able to prepare yellow-seeded transformed plants—such as transformed yellow-seeded Brassica napus.

In the present application the term “transformed” mean cells and plants that do not occur naturally but have, instead, been prepared by human intervention—such as by any one or more of selective cross-breeding and/or biotechnological techniques, preferably a process that includes a biotechnological step (such as chromosome doubling and/or an embryo rescue step).

Thus, according to one broad aspect of the present invention we provide a transformed cell or tissue capable of yielding yellow seeds.

Thus, according to a further aspect of the present invention we provide a transformed plant capable of yielding yellow seeds.

The above mentioned aspects of the present invention will now be described in more detail.

In the text, the term “yellow seed or yellow-seeded” means a visible yellow embryo produced by a substantially transparent seed coat.

In the text, the term “transparent” means a substantial absence of seed coat pigmentation when compared with the wild type seed coat.

In the text, the term “stable” means a yellow seed colour which is consistently trasmitted through subsequent generations.

Preferably, the seed colour is consistently trasmitted through at least two generations, preferably through at least three generations preferably through at least four generations, preferably through at least five generations, preferably through at least six generations, more preferably through at least seven generations.

In the text, the term “consistent” means a substantially even distribution of the yellow colour.

The term “low level” in relation to erucic fatty acid means a level less than 2% erucic acid.

The term “medium level” in reation to erucic fatty acid means a level greater than 2% erucic acid and less than 40% erucic acid.

The term “high level” in relation to erucic fatty acid means a level greater than 40% erucic acid.

The term “low level” in relation to glucosinolate(s) means a level of total glucosinolate(s) less than 25 μmol/g seed.

The term “medium level” in reation to glucosinolate(s) means a level of total glucosinolate(s) greater than 25 μmol and less than 40 μmol per g seed.

The term “high level” in relation to glucosinolate(s) means a level of total glucosinolate(s) greater than 40 μmol/g seed.

In arriving at the present invention, it was recognised that the main constraint of attempting to breed yellow-seeded B. napus plants that could produce yellow seeds was the lack of a transparent seed coat gene. More, in particular, it was recognised that there was an absence of a transparent seed coat gene in the CC genome of B. napus.

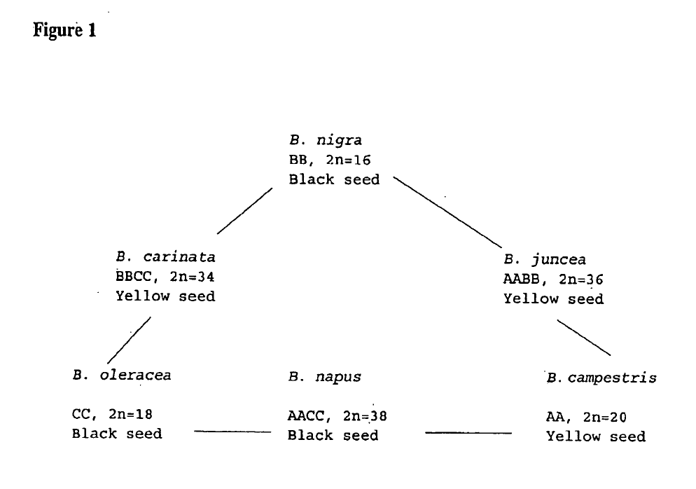

In part of the analysis of the present invention, which is represented by way of FIG. 1, it was recognised that among the diploid Brassica species, yellow-seeded forms can only be found in the B. campestris (AA, 2n=20) species, whereas among the amphidiploid species, yellow-seeded variants can be found in B. carinata (BBCC, 2n=34) and in B. juncea (AABB, 2n=36). The yellow seed in these species is due to a transparent seed coat.

However, in accordance with the present invention it was surprisingly found that it was possible to transfer a transparent seed coat gene into a plant, such as into its genome, that lacked such a gene.

The transfer can occur via biotechnological techniques or by selective cross-breeding procedures which incorporate certain essential or preferred technical features. Details of these techniqties and procedures are presented later on.

Up until the present invention, interspecific crosses in Brassica have been carried out in different directions to exploit the yellow-seeded natural variants of B. campestris, B. juncea and B. carinata with a view to breeding yellow-seeded B. napus. So far, only limited success has been achieved. By way of example, Barcikowska et al. (1987) and Zaman (1988) attempted to develop a yellow-seeded CC genomic species using the interspecific cross of {yellow-seeded B. carinata×black-seeded B. alboglabral B. oleracea} as an intermediate step in breeding yellow-seeded B. napus but in these studies only partly yellow seed was obtained.

Liu (Liu 1983; Liu and Gao 1987) reported on an attempt to breed a yellow seeded B. napus developed from the {B. napus×B. campestris (B. chinensis)} cross. However, the frequent occurrence of black seed and/or black spots on the coat, as well as the change in seed coat colour due to environmental factors, indicated that their yellow seeded strains were not stable over generations.

Zaman (1988) isolated a brown-seeded strain from an interspecific cross of {(B. carinata×B. campestris)×B. napus}. Chen (Chen et al 1988; Chen and Heneen 1992) obtained yellow seed in an F₂ population derived from a cross-between the resynthesised B. napus (resynthesised from B. campestris and a partly yellow-seeded B. alboglabra) and a yellow-brown-seeded B. napus breeding line. However, the yellow seed character did not breed true—such as up to F₄ generation. Chen's partly-yellow-seeded B. alboglabra was developed from an interspecific cross of yellow-seeded B. carinata and black-seeded B. alboglabra.

Rashid et al. (1994) reported on their attempt to develop yellow-seeded plants from a more complex interspecific cross of {[(B. napus×B. juncea)×B. napus]×[(B. napus×B. carinata)×B. napus]}. However, the stability of the seed coat colour in the resulting hybrids was not reported.

Van Deynze (Van Deynze et al. 1993; Van Deynze and Pauls 1994) reported on a partly yellow-seeded B. napus plant which were developed in the University of Manitoba/Guelph. However, an unstable yellow seed colour and a significantly high seed coat pigmentation (black/dark brown seed) was observed when the yellow-seeded strains were grown at a lower temperature of 16° C./12° C. (day/night). This instability of yellow seed colour. may render such varieties unsuitable for some major B. napus growing areas such as Northern Europe and North America.

Tang et al. (1997) developed yellow-seeded B. napus lines from different breeding approaches, such as from interspecies crosses of {(B. campestris×B. oleracea)×B. napus}; {B. napus×B. juncea}; {B.napus×B. campestris}; from irradiated progenies of B. napus; and from progenies of intervarietal crosses of B. napus. However, the stability of the yellow seed colour was not reported.

It is apparent that transparent seed coat gene of B. campestris, B. juncea and B. carinata have been utilised in the prior art in different ways with a view to breeding yellow-seeded B. napus plants.

In contrast, our invention relates inter alia to the transfer of the transparent seed coat gene via the AA genome of Yellow Sarson (B. campestris) into the CC genome of B. napus. Indirect evidence is provided that the transfer of the transparent seed coat gene is achieved through allosyndesis between the A- and the C-genome chromosomes. The resultant B. napus strain, carrying the transparent seed coat gene of Yellow Sarson (B. campestris) in its CC genome, was used in breeding yellow-seeded oilseed rape.

By way of further background information, Attia (Attia and Röbbelen 1986; Attia et al. 1987) studied meiotic configuration of chromosomes in: (i) amphihaploids AC, AB and BC which were generated from crosses between three elementary diploid species and (ii) digenomic triploids AAC, ACC, BBC and BCC which were generated from crosses between the amphidiploid and the diploid species. In the amphihaploids AC 12.3 chiasmata (7.3 II) was found. In AB 5.8 chiasmata (4.36 II) was found and in BC 2.0 chiasmata (1.9 II) was found. In the digenomic triploids BBC and BCC, there was a predominance of preferential pairing between the two homologous genomes while the third single genome remained unpaired. On the other hand, in the AAC and the ACC triploids, a high frequency of allosyndetic pairing between the A and the C genome was expressed by the formation of one or more trivalents in over 50% of the pollen mother cells. According to Busso et al. (1987), the homologous pairing between the A and the C genome chromosomes in trigenomic haploid (ABC) was not disturbed by the presence of the B genome chromosomes.

These cytological observations clearly suggested that the Brassica A and C genome chromosomes are more closely related to each other, while the B genome chromosomes are phylogenetically distant from the A and the C genome chromosomes. Therefore, in amphihaploid genome constitution, it was recognised the transfer of gene(s) from the A- to the C-genome or vice versa could occur relatively easily.

One technique for preparing the transformed plants—namely by use of the AA genome as a vector for the transparent seed coat gene—is presented in the following examples.

As an alternative, transformed plants may be prepared by use of recombinant DNA techniques. By way of one example, the transparent seed coat gene may be transferred to a host plant by use of appropriate vectors—such as by use of Agrobacterium vectors.

By way of another example, in plants where the black seed coat gene is dominant and the transparent seed coat gene is recessive, antisense techniques can be utilised to suppress the dominant black gene and facilitate the expression of the transparent gene.

Even though the transfer of a transparent seed coat gene according to the present invention is not disclosed in EP-B-0470145 and CA-A-2006454, those two documents do provide some usefil background commentary on the types of techniques that may be employed to prepare transformed plant cells and transformed plants according to the present invention. Some of these background teachings are now included in the following commentary.

The basic principle in the construction of genetically modified plants is to insert genetic information in the plant genome so as to obtain a stable maintenance of the inserted genetic material.

Several techniques exist for inserting the genetic information, the two main principles being direct introduction of the genetic information and indirect introduction of the genetic information by use of a vector system. A review of the general techniques may be found in articles by Potrykus (Annu Rev Plant Physiol Plant Mol Biol [1991] 42:205-225) and Christou (Agro-Food-Industry Hi-Tech March/April 1994 17-27).

Thus, in one aspect, the present invention relates to a vector system which carries the transparent seed coat gene according to the present invention and which is capable of introducing the transparent seed coat gene into the genome of an organism, such as a plant.

The vector system may comprise one vector, but it can comprise at least two vectors. In the case of two vectors, the vector system is normally referred to as a binary vector system. Binary vector systems are described in further detail in Gynheung An et al. (1980), Binary Vectors, Plant Molecular Biology Manual A3, 1-19.

One extensively employed system for transformation of plant cells with a given promoter or nucleotide sequence or construct is based on the use of a Ti plasmid from Agrobacterium tumefaciens or a Ri plasmid from Agrobacterium rhizogenes (An et al. [1986], Plant Physiol. 81, 301-305 and Butcher D. N. et al. [1980], Tissue Culture Methods for Plant Pathologists, eds.: D. S. Ingrams and J. P. Helgeson, 203-208).

Several different Ti and Ri plasmids have been constructed which are suitable for the construction of the plant or plant cell constructs described above.

The transparent seed coat gene of the present invention should preferably be inserted into the Ti-plasmid between the border sequences of the T-DNA or adjacent a T-DNA sequence so as to avoid disruption of the sequences immediately surrounding the T-DNA borders, as at least one of these regions appear to be essential for insertion of modified T-DNA into the plant genome.

As will be understood from the above explanation, if the organism is a plant, then the vector system of the present invention is preferably one which contains the sequences necessary to infect the plant (e.g. the vir region) and at least one border part of a T-DNA sequence, the border part being located on the same vector as the genetic construct. Preferably, the vector system is an Agrobacterium tumefaciens Ti-plasmid or an Agrobacterium rhizogenes Ri-plasmid or a derivative thereof, as these plasmids are well-known and widely employed in the construction of transformed plants, many vector systems exist which are based on these plasmids or derivatives thereof.

In the construction of a transformed plant the transparent seed coat gene of the present invention may be first constructed in a microorganism in which the vector can replicate and which is easy to manipulate before insertion into the plant. An example of a useful microorganism is E. coli., but other microorganisms having the above properties may be used. When a vector of a vector system as defined above has been constructed in E. coli. it is transferred, if necessary, into a suitable Agrobacterium strain, e.g. Agrobacterium tumefaciens. The Ti-plasmid harbouring the transparent seed coat gene of the invention is thus preferably transferred into a suitable Agrobacterium strain, e.g. A. tumefaciens, so as to obtain an Agrobacterium cell harbouring the transparent seed coat gene, which DNA is subsequently transferred into the plant cell to be modified.

As reported in CA-A-2006454, a large number of cloning vectors are available which contain a replication system in E. coli. and a selection means which allows a selection of the transformed cells. The vectors contain for example pBR322, the pUC series, the M13 mp series, pACYC 184 etc. In this way, the transparent seed coat gene of the present invention can be introduced into a suitable restriction position in the vector. The contained plasmid is used for the transformation in E.coli. The E.coli. cells are cultivated in a suitable nutrient medium and then harvested and lysed. The plasmid is then recovered and then analysed—such as by any one or more of the following techniques: sequence analysis, restriction analysis, electrophoresis and further biochemical-molecular biological methods. After each manipulation, the used DNA sequence can be restricted or selectively amplified by PCR techniques and connected with the next DNA sequence. Each sequence can be cloned in the same or different plasmid.

After each introduction method of the transparent seed coat gene according to the present invention in plants the presence and/or insertion of further DNA sequences may be necessary. If, for example, for the transformation, the Ti- or Ri-plasmid of the plant cells is used, at least the right boundary and often however the right and the left boundary of the Ti- and Ri-plasmid T-DNA, as flanking areas of the introduced genes, can be connected. The use of T-DNA for the transformation of plant cells has been intensively studied and is described in EP-A-120516; Hoekema, in: The Binary Plant Vector System Offset-drukkerij Kanters B. B., Alblasserdam, 1985, Chapter V; Fraley, et al., Crit. Rev. Plant Sci., 4:1-46; and An et al., EMBO J. (1985) 4:277-284.

Direct infection of plant tissues by Agrobacterium is a simple technique which has been widely employed and which is described in Butcher D. N. et al. (1980), Tissue Culture Methods for Plant Pathologists, eds.: D. S. Ingrams and J. P. Helgeson, 203-208. For further teachings on this topic see Potrykus (Annu Rev Plant Physiol Plant Mol Biol [1991] 42:205-225) and Christou (Agro-Food-Industry Hi-Tech March/April 1994 17-27). With this technique, infection of a plant may be done on a certain part or tissue of the plant, i.e. on a part of a leaf, a root, a stem or another part of the plant.

Typically, with direct infection of plant tissues by Agrobacterium carrying the transparent seed coat gene, a plant to be infected is wounded, e.g. by cutting the plant with a razor or puncturing the plant with a needle or rubbing the plant with an abrasive. The wound is then inoculated with the Agrobacterium. The inoculated plant or plant part is then grown on a suitable culture medium.

When plant cells are constructed, these cells are grown and maintained in a medium following well-known tissue culturing methods—such as by culturing the cells in a suitable culture medium supplied with the necessary growth factors such as amino acids, plant hormones, vitamins, etc, but wherein the culture medium comprises a component according to the present invention. Regeneration of the transformed cells into genetically modified plants may be accomplished using known methods for the regeneration of plants from cell or tissue cultures, for example by selecting the transformed shoots and by subculturing the shoots on a medium containing the appropriate nutrients, plant hormones, etc.

Further teachings on plant transformation may be found in EP-A-0449375.

Reference may even be made to Spngstad et al (1995 Plant Cell Tissue Organ Culture 40: pp 1-15) as these authors present a general overview on transformed plant construction.

In a highly preferred embodiment, the present invention is based on our finding that it is possible to use the transparent seed coat gene of the present invention to prepare transformed cells.

In addition, the present invention also covers transformed plants comprising the transparent seed coat gene of the present invention.

In a preferred embodiment the present invention covers transformed Brassica plants comprising the transparent seed coat gene of the present invention.

In a highly preferred embodiment the present invention covers transformed Brassica plants comprising the transparent seed coat gene obtainable from the Yellow Sarson (Brassica campestris) species.

The following samples were deposited in accordance with the Budapest Treaty at the recognised depositary of The National Collections of Industrial and Marine Bacteria Limited (NCIMB) at 23 St. Machar Drive, Aberdeen, Scotland, United Kingdom, AB24 3RY on 7th December 1998.

Brassica napus 13-217—deposit number NCIMB 40991

Brassica napus 13-219—deposit number NCIMB 40992

The present invention will now be described only by way of example, in which reference is made to the following Figures:

FIG. 1 which is a schematic diagram;

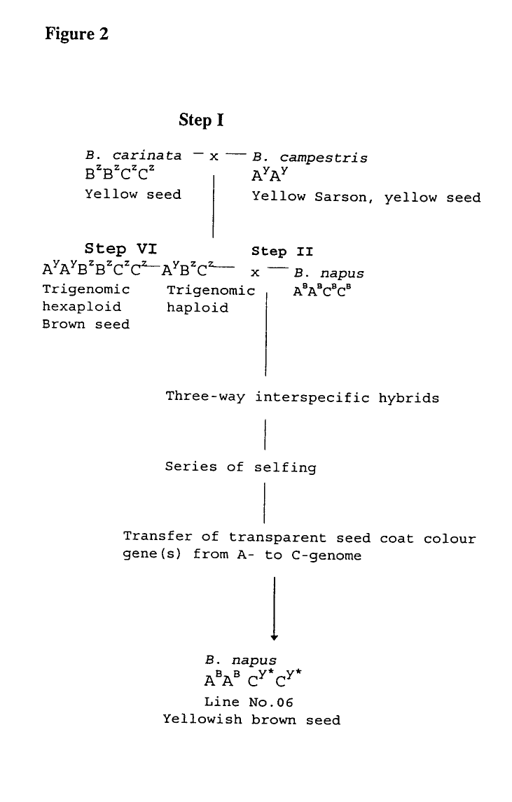

FIG. 2 which is a schematic diagram;

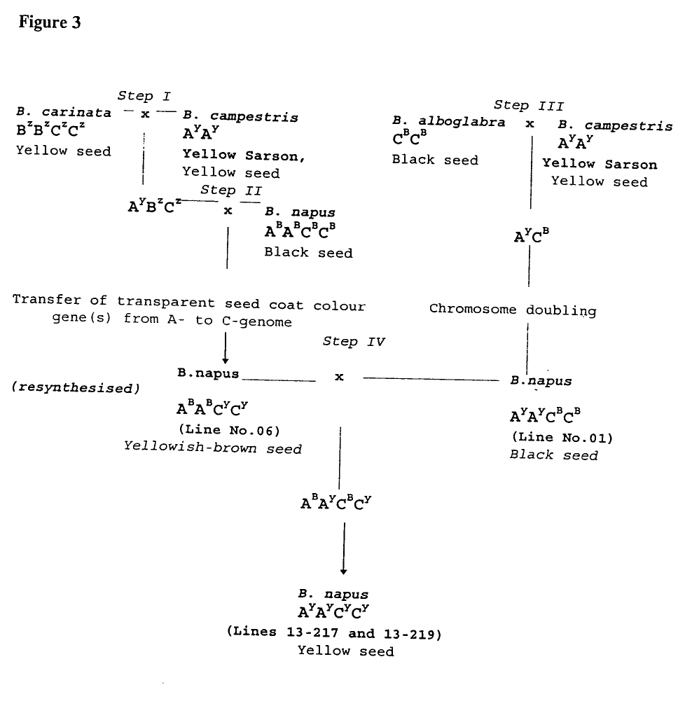

FIG. 3 which is a schematic diagram;

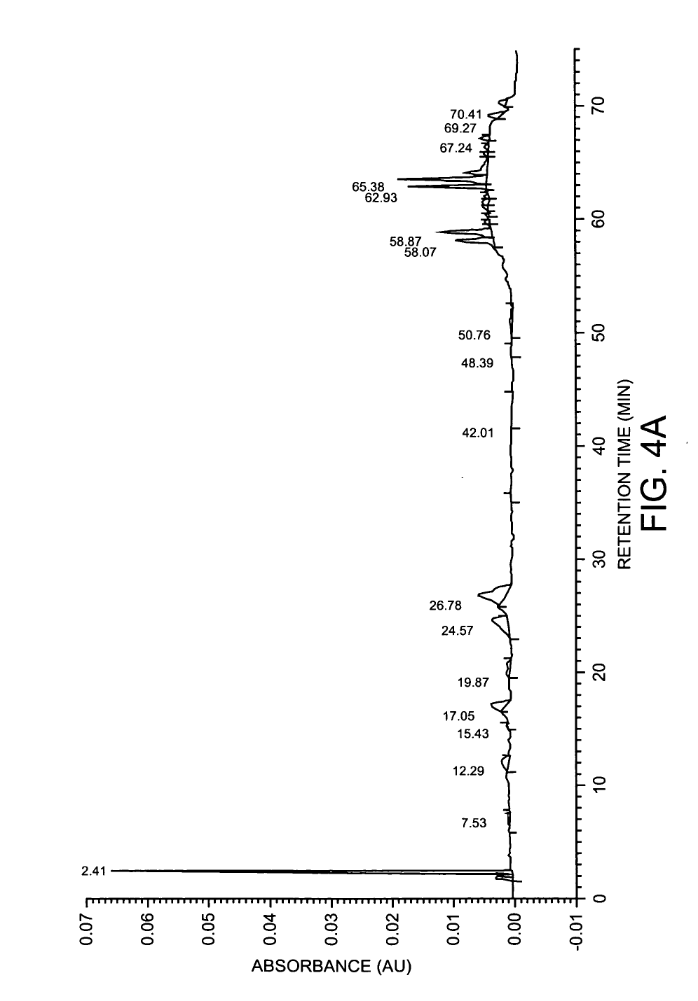

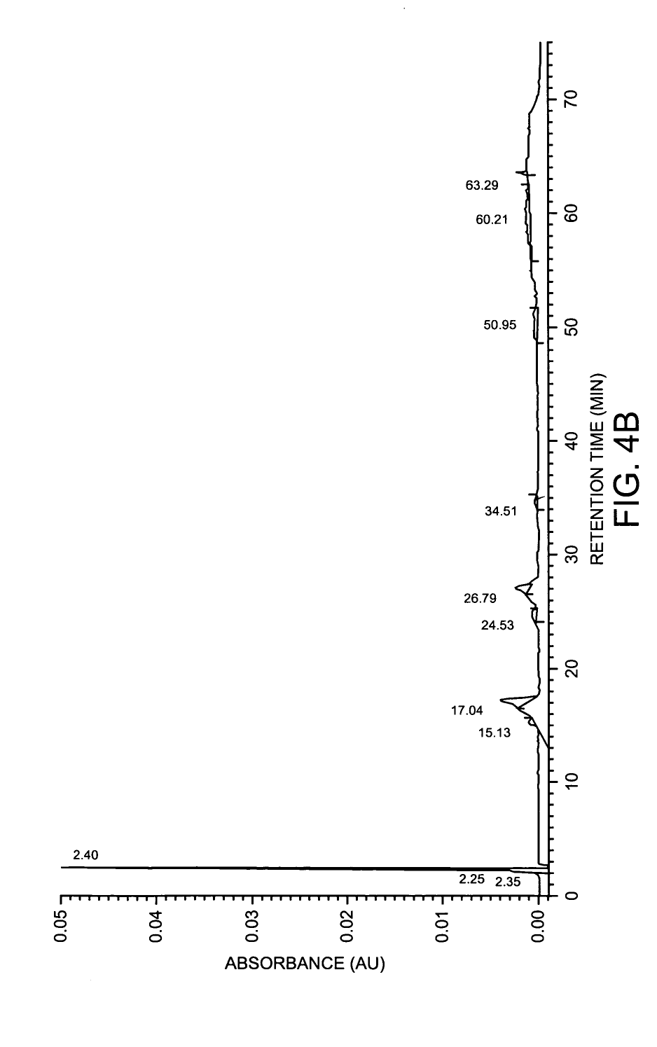

FIG. 4A which is schematic diagram;

FIG. 4B which is schematic diagram;

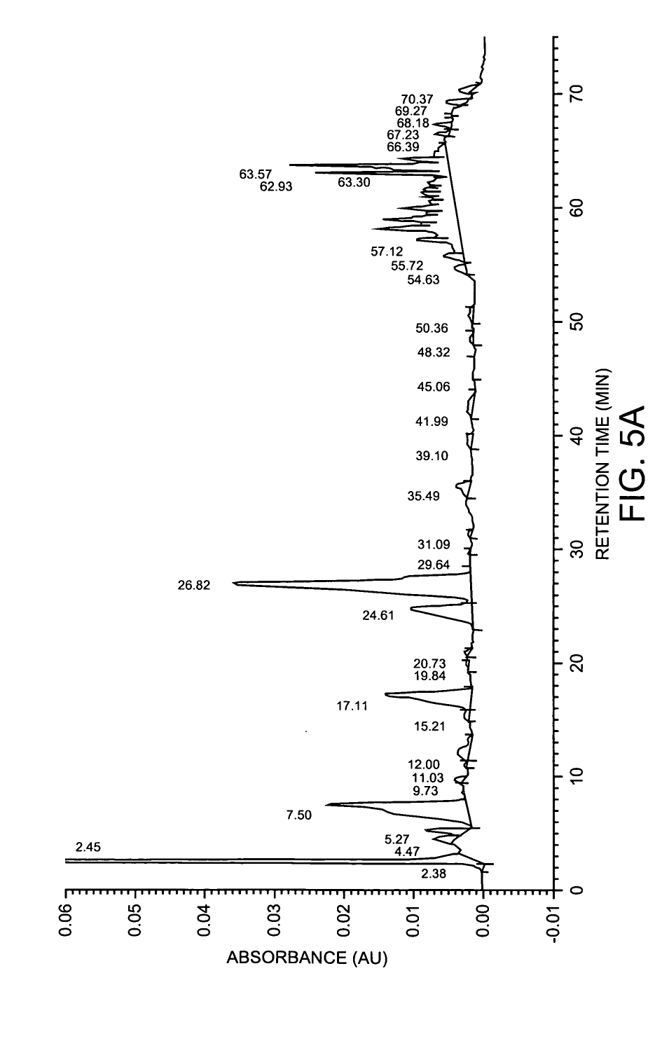

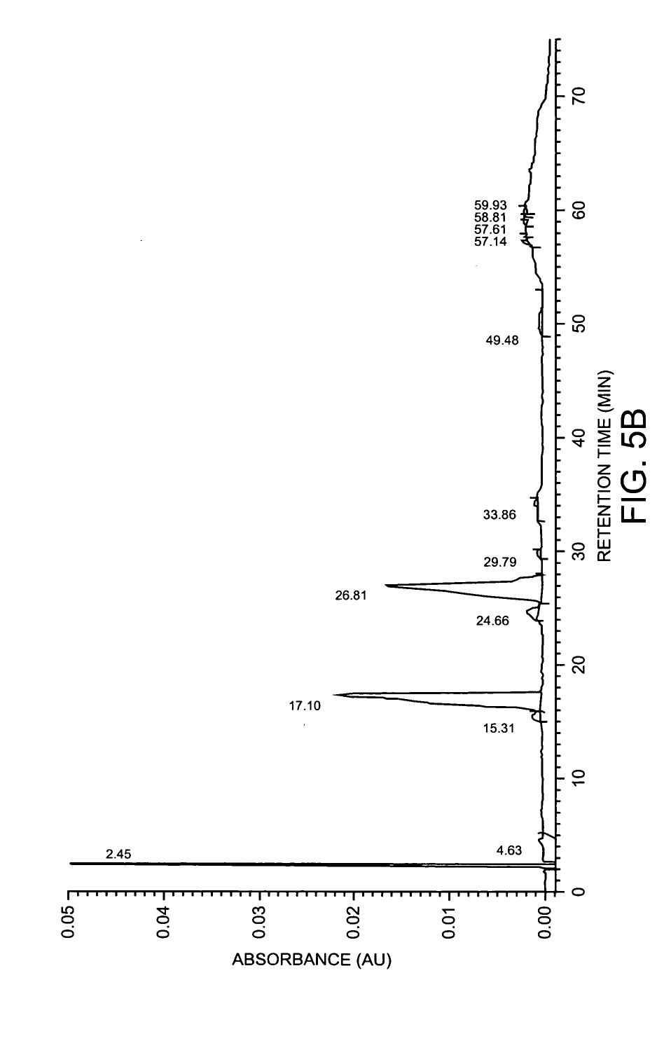

FIG. 5A which is schematic diagram;

FIG. 5B which is schematic diagram;

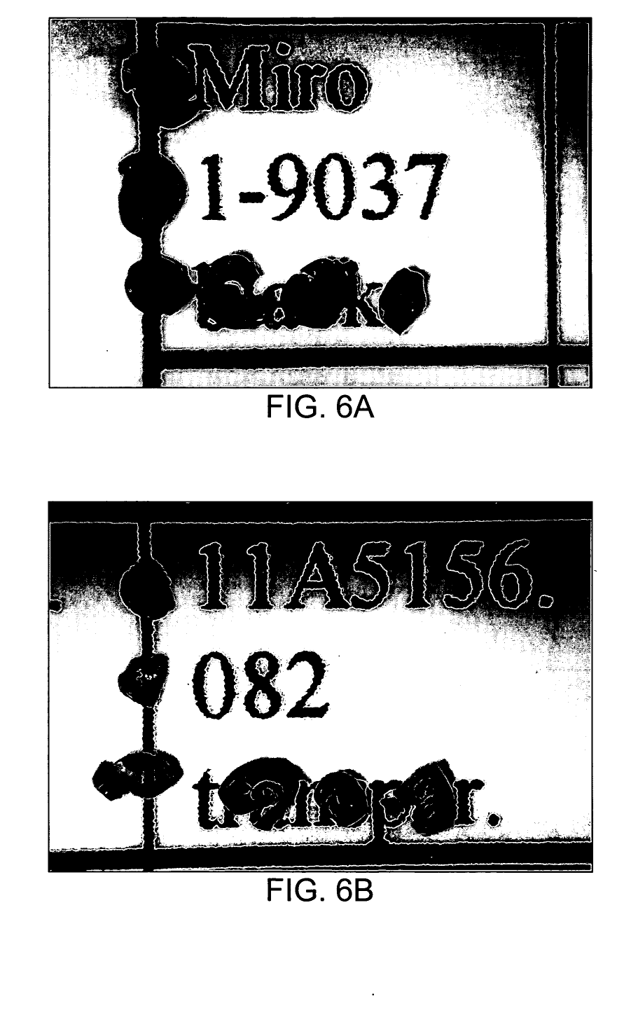

FIG. 6A which is a photographic representation;

FIG. 6B which is a photographic representation;

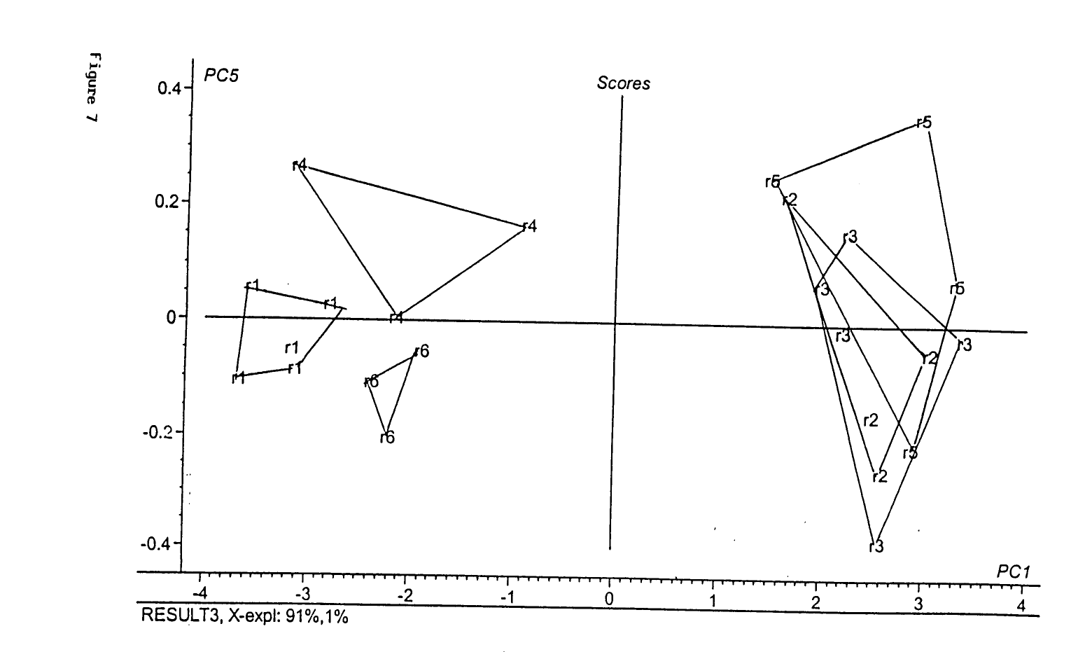

FIG. 7 which is a schematic diagram; and

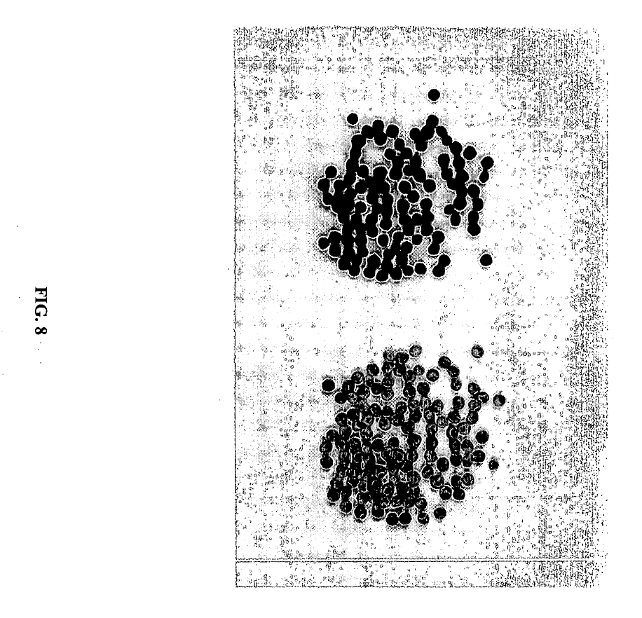

FIG. 8 which is a photographic representation.

In more detail, FIG. 1 is a schematic diagram showing the occurrence of yellow seed in the Brassica species.

FIG. 2 is a schematic diagram showing the approach taken for transferring the transparent seed coat gene from the A- to the C-genome. The normal size capital letters A, B and C indicate the three Brassica genomes. The superscripted letter y indicates the transparent seed coat gene of Yellow Sarson (B. campestris). The superscripted letter z indicates the transparent seed coat gene of B. carinata which are of different genomes relative to the transparent seed coat gene of Yellow Sarson (B. campestris). The superscripted letter B indicates the black seed coat gene of natural B. napus. The symbol * indicates the transfer, through allosyndesis, of the transparent seed coat genes from the A- to the C-genome.

FIG. 3 is a schematic diagram showing the process of development of the yellow seeded B. napus from the interspecific crosses. The genomic and seed coat gene designations are as described in FIG. 2.

FIG. 4A is a HPLC chromatogram of alkaline hydrolysate of transparent seed coat (11A5156.082-K1217) of Brassica napus measured at 280 nm.

FIG. 4B is a HPLC chromatogram of alkaline hydrolysate of transparent seed coat (11A5156.082-K1217) of Brassica napus measured at 360 nm.

FIG. 5A is a HPLC chromatogram of alkaline hydrolysate of black seed coat (Miro) of Brassica napus measured at 280 nm.

FIG. 5B is a HPLC chromatogram of alkaline hydrolysate of black seed coat (Miro) of Brassica napus measured at 360 nm.

FIG. 6A is a photographic representation of seed coats from black seeds of B. napus.

FIG. 6B is a photographic representation of seed coats from yellow seeds of B. napus. The yellow seeds have a transparent seed coat.

FIG. 7 is a Principal Component Analysis (PCA) plot of Agrovision colour data from six seed samples (dark and yellow seeds) of Brassica napus.

FIG. 8 demonstrates the colour difference between yellow seed (from the present invention) and black seed (conventional type) of Brassica napus.

Methods and Procedures

The complete procedure of the present invention is given stepwise in Steps I-VI.

Steps I-II (which are summarized in FIG. 2) involve the generation of yellowish brown seeded B. napus plants carrying the transparent seed coat genes of Yellow Sarson (B. campestris) in its CC genome.

Step III (which is summarised in FIG. 3) involves the resynthesis of the B. napus line carrying the transparent seed coat genes of Yellow Sarson (B. campestris) into its AA genome.

Step IV (which is also summarized in FIG. 3) involves the development of the yellow seeded B. napus using the materials developed in Step I-II and Step III.

Step V involves the development of yellow-seeded B. napus of double low quality by crossing yellow-seeded lines with conventional B. napus varieties of double low quality.

Step VI involves the generation of hexaploids which supports the claim of the present invention.

Step I: Interspecific Cross

Yellow-Seeded B carinata×Yellow-Seeded Yellow Sarson (B. campestris)

An interspecific cross between the yellow-seeded B. carinata (BBCC) line (Accession No. 381078) and the yellow-seeded Yellow Sarson (B. campestris) (AA) line of Bangladesh origin (Accession No. 3-0166.001) was carried out and trigenomic haploid plants (ABC) were generated (FIG. 2).

Step II: Interspecific Cross

Trigenomic F₁ Hybrid (ABC)×Black-Seeded B. napus (AACC)

Trigenomic haploids (ABC) of Step I were crossed with black-seeded natural B. napus (Accession No. 1-9007) and the resultant three-way interspecific hybrids were selfed for a number of generations (FIG. 2). These interspecific crosses were designed to ensure allosyndesis between the A- and the C-genome chromosomes in the trigenomic haploids (ABC) and subsequent selfed generations would transfer the transparent seed coat gene from the A- to the C-genome.

Steps I-II resulted in the yellowish-brown seeded B. napus line No. 06.

Step III: Interspecific Cross to Resynthesise B. napus

(Black-Seeded B. alboglabra×Yellow-seeded Yellow Sarson (B. campestris)

An interspecific cross between black-seeded B. alboglabra (CC) (Accession No. 381053) and yellow-seeded Yellow Sarson (B. campestris) (AA) (Accession No. 381049) was carried out to resynthesise B. napus (AACC) (FIG. 3). As the reciprocal crosses failed to produce any viable hybrid seeds in vivo, the application of an embryo rescue technique (described below) followed by in vitro culture was used to obtain hybrid plants.

Step III resulted in the black-seeded B. napus line No. 01.

Step IV: Generation of Yellow-Seeded B. napus

(Yellowish-Brown-Seeded B. napus (No. 06)×Black-Seeded Resynthesized B. napus (No. 01))

A conventional crossing were made between the lines No. 06 and No. 01 (FIG. 3) and pedigree breeding was followed. In the F₆ generation, complete yellow seeded lines were obtained.

Step IV resulted in yellow-seeded B. napus lines 13-217 and 13-219.

Step V: Development of Yellow-Seeded B. napus of Double Low Quality (Zero Erucic Acid in Seed Oil and Low Glucosinolate(s) in Seed Meal)

In order to develop a yellow-seeded B.napus line of double low quality (zero-erucic acid and low glucosinolate(s)), yellow-seeded B. napus lines (13-217 and 13-219) were crossed with conventional B. napus varieties of double low quality such as Polo and Dakini. A microspore culture technique (Lichter 1989) was applied on all resulting F₁ plants to generate doubled haploid (DH) lines. One zero erucic acid yellow-seeded DH line was crossed with the two (Polo and Dakini) conventional B. napus varieties of double low quality to develop yellow-seeded B. napus of double low quality. Pedigree breeding was followed for this purpose.

Step VI: Generation of Trigenomic Hexaploids

(Yellow-Seeded B. carinata×Yellow-Seeded Yellow Sarson (B. campestris)

In order to assess the joint action of the transparent seed coat genes of B. carinata (BBCC) and Yellow Sarson B. campestris (AA) on seed coat pigmentation, fertile trigenomic hexaploids (AABBCC) were generated by chromosone doubling of trigenomic haploids (ABC) from {yellow-seeded B. carinata×yellow-seeded Yellow Sarson (B. campestris)} plants of Step I.

Embryo Rescue Technique

The steps for the embyo rescue technique were as follows:

(i) the siliquae were harvested at the correct stage of development;

(ii) the green embryos (survived embryos) were rescued and were cultured in vitro; and

(iii) the survived embryos were sub-cultured for shoot regeneration.

In a particular detailed embodiment, B. alboglabra plants were cross pollinated with pollen from Yellow Sarson (B. campestris) and immature siliquae at 24 to 28 days after pollination were harvested, excised and the hybrid embryos were rescued using the following sterile and cell culture procedures. Excised siliquae were surface sterilised with 70% ethanol for three minutes followed by treatment with a 5% calcium hypochloride solution for 20 minutes. Siliquae were washed three times with sterile water and logitudinally dissected under a stereo microscope. The developed (fertilised) ovules were excised and obtainable embryos were rescued. These rescued embryos were cultured at 24° C. in Murashige and Skoog's (1962) medium supplemented with 0.1 mg NAA and 0.1 mg Kinetin per litre of medium and under a continuous illumination of 806 lux. After 20-30 days of culture, survived embryos were sub-cultured in the same medium for shoot induction.

Chromosome Doubling

(a) Chromosome Doubling of in vitro Culture Regenerated Shoots

The essential steps for the chromosome doubling technique were as follows:

(i) the shoots were excised and were submerged in an aqueous solution of colchicine;

(ii) after treatment with colchicine, the shoots were washed with sterile water and the lower part of the stem was removed by cutting; and

(iii) the shoots were transferred into rooting media.

In a particular detailed embodiment, chromosome doubled plants were obtained by treating regenerated shoots at 2-4 leaf stage (unfolded leaf) with an aqueous solution of 0.5% colchicine plus 2.5% DMSO (modified version of Gland (1981). Shoots were submerged in this solution up to the level of the meristem/shoot apex. After 16-20 hours, shoots were taken out, washed with sterile water for three times and the 3-5 mm lower parts of the shoots were cut off. Shoots were then transferred into Murashige and Skoog's (1962) medium supplemented with 3 mg IBA per litre of medium for root induction and were placed under a continuous illumination of 800 lux.

(b) Chromosome Doubling of Plant Branches in vivo

The essential steps for the chromosome doubling technique were as follows:

(i) the leaf exils was injected with an aqueous solution of colchicine;

(ii) repeat injection were administered in the same place at intervals.

In a particular detailed embodiment, chromosome doubled trigenomic hexaploid branches/shoots were obtained by injecting trigenomic haploids (ABC) with an aqueous solution of 0.5% colchicine plus 2.5% DMSO (modified version of Gland 1981). Specifically, 38 leaf axils from 19 plants were injected three times with the colchicine/DMSO solution at 24 hour intervals. The hexaploid nature of the branches was confirmed by visual observation as well as by flow-cytometric analysis of nuclear DNA. These branches produced fertile flowers, that is, they had viable pollen and on selfing produced viable seeds, in contrast to the flowers of the other branches (trigenomic haploid) which were sterile.

Flow Cytometric Analysis

Flow-cytometric analysis was carried out following the methods described by Galbraith et al. (1983) and Sabharwal and Dolezel (1993). For this purpose, nuclear DNA from seedlings raised from selfed seeds were used.

Isozyme Electrophoresis

Leaves (approximately 3 cm in size) from three-week-old plants were homogenised in 0.1M Tris-HCl buffer (pH 7.2) containing 0.5% DDT (1-4 Dithio-DL-threitol) and centrifuged at 20,000 rpm for 1.5 minutes after which the supernatants of the samples were adsorbed on 9 mm×3 mm filter paper (Whatman No. 1). A horizontal starch gel (13% starch hydrolysed, Sigma S-4501) was prepared and-the samples were applied to cover approximately ⅓ of the gel-width from the cathodal end of the gel. The gel and tray buffer systems were 5 mM L-Histidine-mono-hydrochloride (adjusted with NaOH to pH 7.0) and 0.4 M tri-sodium-citrate-di-hydrate 0.1 M citric acid, (pH 7.0) respectively. The electrophoresis was carried out for 3.5 hours at 200 V, 150 mA at 0° C. to ensure an adequate separation of the enzymes. All subsequent staining procedures were carried out according to Murphy et al. ((1990).

Erucic Acid Measurement

Erucic acid content in seed oil was measured by gas chromatographic analysis following the method described by Association of Official Analytical Chemists (1990). Typically, seed oil (triglyceride of fatty acids) was extracted from ground seeds with hexane and methylated by addition of methanol. The methylesters of fatty acids were analysed by gas chromatography using a HP5890 Series II instrument supplied with a FID detector and autosampler coupled with ChemStation HP3365. The column was a 50 m long and 0.32 mm wide WCOT fused silica (Chrompack) with a stationary of CP-sil-58CB. Helium was employed as carrier gas at a flow rate of 276 m/min. An oven temperature of 205° C., an injection temperature of 250° C. and a detection temperature of 270° C. were used.

Seed Glucosinolate(s) (GLS) Measurement

A quantitative measurement of total seed glucosinolate(s) (GLS) was carried out using the method described by Smith et al. (1985). A qualitative measurement of seed GLS levels, using the Glucose-Test method, was carried out as follows: Two hundred microlitres of distilled water was added to five crushed seeds and incubated at room temperature for ten minutes. A Medi-Test Glucose strip (manufactured by Macherey-Nagel) was inserted into the solution and the colour change which appeared after thirty to sixty seconds was rated on a scale of one to five.

Chemical Fingerprinting of Transparent Seed Coats Compared to Black Seed Coats of B. napus

Seed coats were analysed essentially according to the method of Andreasen et al. 1998 (Journal of the Science of Food and Agriculture, in press) for the extraction of soluble and insoluble esterbound phenolic acids. Seed coats of line 11A5156.082-K1217 (transparent seed coat) (FIG. 6B) and the variety Miro (black seed coat) (FIG. 6A) were prepared. manually and further purified by a gravimetrically method employing a vertical air stream. The seed coats (100 mg) were homogenised using a mortar, added 100 ml 1.0 M NaOH and incubated at room temperature for 18 hours under N₂ in the dark. Then the pH of the hydolysates was adjusted to below 2 with 1.0 M HCl, extracted 3 times with 40 ml ethylacetate. The combined ethylacetate fractions were evaporated under vacuum at room temperature and the dried residue was dissolved in 3.0 ml of 50% acetonitrile and 2.0% trifuoroacetic acid and filtered through a 0.45 μm nylon filter.

The extracts were analysed by HPLC (Hitachi, Merck) equipped with an analytical RP-18 LiChroCART250-4 (5 μm) column at 35° C. using a gradient solvent system consisting of solvent A (50% acetonitrile and 0.5% trifluoroacetic acid) and Solvent B (8% acetonitrile and 0.5% trifluoroacetic acid) with the following elution profile: 0-10 min: 100% B, 11-20 min: 90% B, 21-30 min: 85% B, 31-45 min: 80%, 46-50 min: 75% B, 51-65 min: 100% A and 66-80 min: 100% B. Flow rate: 1 ml/min. Injection volume: 50 μl. Detection with a photodiodearray detector at 280 nm and 360 nm.

Image Analysis

Six different seed samples were used for this purpose:

| Sample number | Seed colour |
|---|---|
| 1 | Black |
| 2 | Yellow |
| 3 | Yellow |
| 4 | Black |
| 5 | Yellow |
| 6 | Dark Brown |

Image analysis was done with Agrovision Grain Check where reflection of red, green and blue colour light as well as total light reflection was measured. Data were analysed using multivariate analysis system, where only Principal Component analysis (PCA) was done.

Results

Step I

Interspecific Cross {B. carinata×Yellow Sarson (B. campestris)}

When the B. carinata line was used as the female in the interspecific cross, 3.28 hybrid seeds per pollination were obtained. The reciprocal cross yielded 5.15 seeds per pollination. Although the degree of interspecific crossability between these two species was adequate, a greater number of hybrids (approximately 66%) were obtained by the application of an embryo rescue technique. Thus, the use of the embryo rescue technique for this purpose is a highly preferred feature of the present invention.

Morphology of the Trigenomic (ABC) F₁ Hybrid Plants

The trigenomic F₁ hybrids were easily distinguished from their parental lines by their morphological characteristics. For example, the leaf hairiness, leaf base clasping and the. yellow petal colour of the F₁ hybrids was comparable to the B. carinata line whereas the leaf margin dentation was comparable to that of Yellow Sarson (B. campestris). The confirmation of these interspecific hybrids was carried out using the isozyme electrophoretic procedure as already described.

Step II

Crossability of the Trigenomic F₁ Hybrids (ABC) with B. napus (AACC)

Haploid genome composition of the trigenomic hybrids conferred very low fertility. On crossing with conventional B.napus, the hybrid plants produced approximately 0.025 hybrid seeds per pollination. Almost the same number of fertilised ovules were aborted before reaching to full maturity.

Fertility of the Hybrids Derived from the ABC×AACC Interspecific Cross

The pollen fertility of the hybrids, generated from crossing the trigenomic haploids (ABC) with natural B. napus (AACC), was only 16.6%. As a result, the manual selfing of individual flowers, whereby pollen from fully developed flowers was placed on the stigma, was necessary to harvest a sufficient number of F₂ seeds. All harvested seeds had a black seed coat.

Selection for Seed Coat Colour from the ABC×AACC (F₂ to F₇ Generations)

Two hundred and seventy two F₂ plants were raised (Table 1). The pollen fertility in these plants varied from 0% to 79%. In order to avoid any cross-pollination, the floral raceme of all of the F₂ plants were isolated individually by parchment paper bags. One hundred and six (39%) of the F₂ plants produced seeds using this bag isolation procedure. Of these seed bearing F₂ plants, 97 had a black seed coat while 9 had a brown seed coat. One hundred and sixteen F₃ plants were raised from the 9 F₂ brown seed families of which 99 plants produced viable seeds under bag isolation. One single F₃ family (No. 0026.002) segregated for black, brown and light brown seed colour while the other 8 families produced only black/brown coloured seeds. The light brown coloured F₄ seeds produced on 2 F₃ plants were used for further selection but an improvement in seed coat colour, in terms of obtaining a yellowish brown seed, was only obtained from the progeny of 1 (No. 0026.092) of the 2 F₃ plants. The progeny of the yellowish brown coloured seed could not be completely stabilised in the following F5 and F6 generations. However, the yellowish-brown-seed coloured F₇ line (No. 06) was crossed with the resynthesised B. napus (resynthesised from B. alboglabra×Yellow Sarson (B. campestris) line (No. 01) with a view to developing a true breeding yellow-seeded B. napus plant.

Step III: Interspecific Cross to Resynthesise B. napus

(Black-Seeded B. alboglabra×Yellow-Seeded Yellow Sarson (B. campestris)

An interspecific cross between black-seeded B. alboglabra (CC) and yellow-seeded Yellow Sarson (B. campestris) (AA) was carried out to resynthesise B. napus (AACC) (FIG. 3). As the reciprocal crosses failed to produce any viable hybrid seeds in vivo, an embryo rescue technique (described above) was applied followed by in vitro culture. Thus, the use of the embryo rescue technique for the resynthesis of B. napus is regarded as an essential technical feature of the present invention. For this purpose, B. alboglabra was cross pollinated with Yellow Sarson (B. campestris) and approximately 0.45 embryos per cross pollinated siliquae were rescued of which 40% survived under in vitro culture conditions and gave rise to amphihaploid (AC) plantlets. The plantlets were treated with aqueous solution containing 0.2% colchicine plus 2.5% DMSO for doubling chromosomes. The chromosome doubled resynthesized B. napus was self-compatible in nature and had the white petal characteristic of the B. alboglabra line and on selfing produced black coated seeds.

Step IV: Generation of Completely Yellow-Seeded B. Napus

(Yellowish-Brown-Seeded B. napus (No. 06)×Black-Seeded Resynthesized B. napus (No. 01))

A conventional crossing were made between the lines No.06 and No.01. The low (approximately 10 seeds) yield of seeds per pollination (Table 2) was expected as both of the parental lines were developed through the interspecific crosses already described.

Of the selfed seeds of 259 F₂ plants, which were visually examined for colour (Table 3), 199 (76.8%) produced black/brown coat coloured seeds, 51 (19.7%) produced partly-yellow coloured seeds and 9 (3.5%) produced yellow-brown coloured seeds. F₃ families were generated from the selfed seeds of all the 9 yellow-brown seeded F₂ plants. Of the 9 F₃ families, 4 families segregated for black/brown and partly yellow seeds in a proportion of 19:41, 4 families segregated for black/brown, partly-yellow and yellow-brown seeds in a proportion of 19:48:62 and 1 F₃ family segregated for partly-yellow, yellow-brown and nearly yellow seeds in a proportion of 9:18:1. Thus, 1 nearly yellow-seeded F₃ plant was obtained from a total of 217 F₃ plants from 9 families. Selfed seeds of the single nearly yellow-seeded and selected 8 yellow-brown seeded F₃ plants were used to grow F₄ families. No yellow-seeded plant was found in the F₄ families which were generated from the yellow-brown seeded F₃ plants. On the other hand, the F₄ family generated from the nearly yellow-seeded F₃ plant segregated again for partly-yellow, yellow-brown and yellow seeds. The proportion of yellow seeded plants (40.9%) in this generation was significantly higher than that obtained in the F₃ generation. The F₅ and F₆ generation families were grown only from the selected yellow-seeded plants. Segregation for yellow-brown and yellow seed colour continued with dominance of the yellow-seeded plants in each successive generation. All of the seeds obtained in the F₇ generation had a consistent and stable yellow colour.

Because the yellow-seeded lines thus developed are derived from a series of interspecific crosses, a low fertility in these plants could be expected. Selection for improved fertility was performed along with selection for a consistent and stable yellow seed colour as follows: The yellow-seeded F₄ to F₉ generation plants were crossed with natural B. napus using the yellow-seeded plants as the female. The extent of crossability was increased in each successive generation from 3 seeds per pollination in the F₄ generation almost 7 seeds per pollination in the F₉ generation (Table 4).

The seed oil and seed meal of the yellow-seeded B. napus obtained in the present case contained an intermediate level of erucic fatty acid (26-28%) and a high level of glucosinolate(s) (>40 μmol/g seed), respectively. Such two lines (13-217 and 13-219) were crossed with conventional oilseed rape B. napus of double low quality in order to develop yellow seeded oilseed rape B. napus of double low quality. The above-mentioned two yellow-seeded B. napus lines had white petal, apparently derived from the C-genome of B. alboglabra.

Step V: Development of Yellow-Seeded Oilseed Rape B. napus of Double Low Quality

(Zero Erucic Acid in Oil and Low Glucosinolate(s) in Seed Meal)

F₁ hybrids from crosses between yellow seeded lines (13-217 and 13-219) and conventional B. napus of double low quality such as cultivars Polo and Dakini were used to produce doubled haploid (DH) plants/lines. Two hundred and eighty seven DH plants/lines were generated from four crosses (Table 5).

Inheritance of Seed Coat Colour in Doubled Haploid (DH) Population

The following segregation pattern for seed colour was found in 287 DH lines (Table 5): 195 DH lines had black/dark-brown-seed colour; 34 were reddish-brown, 27 were partly-yellow, 14 were yellow-brown and 17 yellow-seeded. When the data from the black/dark brown, reddish brown, partly yellow and yellow-brown seed coloured DH lines were pooled into one class, a 270:17 distribution pattern was obtained which fits in well with a 15:1 segregation pattern for four gene loci. In contrast, when the data from the black/dark brown, reddish brown and partly yellow seed coloured DH lines were pooled into one class and the data from the yellow-brown and yellow seed coloured DH lines were pooled into a second class, a distribution pattern of 256:31 which fits with a 7:1 segregation pattern for three gene pairs was obtained. It would only be logical to pool the data from the latter two classes (yellow and yellow-brown classes) if the parental yellow-seeded lines used in the crosses would in any instance produce yellow-brown seeds. Selfing 30 plants of these two parental yellow-seed coloured lines grown in the field indicated an instability for seed coat colour in 30% of the plants examined. However, such instability was not found when the plants were grown in glasshouse. Although the chi-square values obtained were not significant for both the four and the three-gene loci model, a better fit to the segregation ratio (in terms of chi-square and p-values) was found with a four-gene loci model indicating that the transparent seed coat colour for yellow seed is due to a homozygous recessive condition in all four loci. As the seed coat colour in B. campestris is governed by two loci and the transparent seed coat colour (yellow seed) is due to homozygous recessive condition in both loci (Stringam 1980, Schwetka 1982), the results suggest that a four gene model for the transparent seed coat colour in B. napus is the most appropriate to consider.

Inheritance of Petal Colour and Erucic Acid Content in Doubled Haploid (DH) Lines

A total of 61 doubled haploid lines from four crosses were examined for petal colour and erucic acid content (Table 6). Twenty six (26) lines had a yellow petal colour and thirty five (35) lines had a white petal colour. This 26:35 distribution ratio agrees with a 1:1 Mendelian segregation pattern (X²=1.05, p=0.5-0.3). Seeds of 27 DH lines were almost free from erucic acid (range of 0.04-3.1%, mean of 0.4%) while 34 lines had an erucic acid content ranging from 15.8-33.4% (mean of 22.7%). This 27:34 distribution ratio is in accordance with a 1:1 Mendelian segregation (X²=0.59, p=0.5-0.3) indicating that a single pair of functional alleles for erucic acid content was present in the two yellow-seeded lines. All of the white petal flowered lines were associated with the presence of erucic acid, except for two lines (0.5 and 3.1% erucic acid) which were almost free from erucic acid. Similarly, all yellow-flowered lines were associated with the absence of erucic acid, except for one line which contained 25.3% erucic acid. These results suggests that the gene locus controlling the petal colour and the locus controlling erucic acid content in the C-genome are located on the same chromosome but that recombination between these two loci can sometimes occur, which in present case, was in 4.9% of the DH lines.

Inheritance of Total Glucosinolate(s) (GLS) Content in Doubled Haploid (DH) Lines

Using the method of Smith et al (1985), seeds of a total of 202 DH lines from the four crosses were examined for GLS content (Table 7). Seeds from 7 lines had less than 20 tmol GLS per g seed while seeds from the remaining 195 lines had greater than 20 μmol GLS per g seed. This 7:195 distribution clearly agrees with the 1:15 segregation pattern for four gene loci. The segregation pattern in each individual cross did not depart significantly from the overall pattern.

From the above mentioned doubled haploid breeding progranmue, it was possible to obtain a yellow seed coloured B. napus line (154.004) whose seed is free from erucic fatty acid and whose seed meal contained a high level of GLS. In order to lower the seed GLS level of the yellow-seeded B. napus, the DH line (154.004) was crossed with conventional oilseed rape of double low quality such as Polo and Dakini. F₂ populations were raised in field plots. Seeds of a total of 144,914 open pollinated F₂ plants were examined for seed coat colour from which open pollinated seeds of 640 plants were harvested as being yellow-seeded or nearly yellow seeded.

The Glucose-Test method was applied for qualitative measurement of GLS content in seeds of these 640 yellow-seeded plants. Using the Glucose Test results, seeds of 507 plants which displayed a very high test score (3-5), were deemed as having a high GLS level and were discarded. A quantitative GLS measurement was carried out on the seeds from the remaining 133 plants that had a test score of 2. Seeds from 12 plants, having relatively low GLS content (25-40 μmol/g seed), were used to develop F₃ generation plants. A total of 600 F₃ plants were grown, selfed and examined for seed colour. Two hundred eighty seven plants produced yellow or yellow-brown or partly-yellow coloured seeds while the remaining 313 plants produced brown or black coloured seeds. The Glucose-Test was carried out on seeds from the above-mentioned 287 plants from which 87 yellow, 24 yellow-brown and 23 partly-yellow seed coloured families were selected for developing F₄ generation plants. Of the 87 F₄ families descended from the yellow-seed coloured F₃ plants, 40 families were stable for yellow seed colour, while 47 F₄ families again segregated for yellow and yellow-brown seed colour. As expected, segregation for seed colour was also observed in the progenies of 24 yellow-brown- and 23 partly-yellow-seed coloured F₃ plants. Selfed seeds from a total of 402 F₄ plants were examined for seed colour and 114 were discarded due to brown seed. Quantitative measurement of GLS was carried out on selfed seeds from the remaining 288 F₄ plants (yellow +yellow-brown+partly-yellow seed). Seeds from five plants were shown to have a GLS level less than 20 μmol/g seed and were yellow in colour.

These results indicate that yellow-seed B. napus may be developed for commercialisation with varying levels of seed oil and seed meal quality such as: (i) zero erucic acid and low glucosinolate(s), (ii) zero erucic acid and high glucosinolate(s), (iii) high erucic acid and low glucosinolate(s), (iv) high erucic and high glucosinolate(s).

Step VI: Synthesis of the Trigenomic Hexaploid (AABBCC) from the B. carinata×Yellow Sarson (B. campestris) Interspecific Cross and its Seed Coat Colour

Trigenomic hexaploids were obtained by injecting trigenomic haploids (ABC) with an aqueous solution of 2.5% DMSO plus 0.2% colchicine which induces chromosome doubling (FIG. 2). The 38 leaf axils from 19 plants which were injected with the colchicine/DMSO solution yielded 18% fertile trigenomic hexaploid shoots (branches) which produced 5.4 to 35.0% viable pollen and were self-compatible. Using manual selfing, an approximately 93% siliqua set was obtained and the number of viable seeds harvested per selfing was 3.27. All selfed seeds were uniform brown in colour. The next generation of plants were grown from selfed seeds and the hexaploid nature of the plants were confirmed by flow-cytometric analysis of nuclear DNA. Selfed seeds produced on these plants were also brown.

Chemical Fingerprinting of Transparent Seed Coats Compared to Black Seed Coats of B. napus

In order to establish whether the transparent appearance of the seed coats is due to a reduction of the concentration of chromogenic substances compared to black seed coats, chemical fingerprinting on HPLC of alkaline seed coat hydrolysates were conducted (FIGS. 4-5).

The chromatogrammes showed that some light absorbing substances were present in much lower quantities in transparent seed coats compared to black seed coats. For example, a substance eluting with a retention time of 7.50-7.53 min was present in a 35-fold lower concentration in the transparent seed coats than in the black seed coats, measured at 280 nm (FIGS. 4A-5A). On average, the transparent seed coats contained 4.6-fold lower concentrations of substances absorbing at 280 nm.

Similar data were obtained by measuring the absorbance at 360 mn simultaneously (FIGS. 4B-5B). For example, two compounds eluting at 17.04-17.11 and 26.78-26.82 min were found in about 8-fold lower concentrations in the transparent seed coats compared to the black seed coats, respectively. As these two compounds displayed significant absorption at both 280 nm and 360 nm, it is possible that they are flavonoids as the group of substances with this characteristic absorption are often associated with colorization of plant tissues and are preferentially extracted by the employed analytical method (Norbaek et al. 1998. Phytochemistry, in press).

Image Analysis

PCA-plot of Agrovision colour data on the 6 seed samples is shown in FIG. 7, where the first principal component (x-axis) is for colour intensity which explained 91% of the recorded total variation among the 6 seed samples. The present data clearly distinguish the yellow seeds samples (No. 2, 3 and 5) from the remaining black (No. 1 and 4) and brown (No. 6) seeds samples. Within the dark seed samples slight variation was found while no variation exists between the three yellow seed samples.

Oil, Protein and Fibre Content in Yellow-Seeded B. napus

Materials and Methods

Two yellow-seeded F₇ lines, 13-217 and 13-219, were crossed with two black-seeded spring type B. napus cvs. ‘Polo’ and ‘Dakini’. Microspore culture technique (Lichter 1989) was applied for production of DH lines from F₁ plants. Bulk seed samples of black, brown and yellow coloured seeds were prepared using a small amount of seeds from these three seed colour type DH lines and were used for measurement of oil, protein and fibre content. Oil and protein content was measured by near infrared reflectance, using a suitably calibrated InfraAlyzer2000 (Bran+Luebbe) and fibre content was measured following the method described by Stringam et al. (1974).

Results

Oil, protein and fibre data from bulk seed samples of black-, brown- and yellow-seeded DH lines are presented in Table 8. The yellow seed sample had higher oil+protein contents

Table 8: Oil, protein and fibre content (in bracket, relative values) in different coloured bulk seed samples of doubled haploid lines from yellow-seeded×black-seeded B. napus cross.

| | Oil + protein | Fibre | Fibre |
|---|---|---|---|
| Seed colour | % seed DM* | % seed | % oil-free meal |
| Black seed | 68.2 (100) | 8.6 (100) | 13.6 (100) |
| Brown seed | 69.8 (102) | 5.9 (69) | 9.6 (71) |
| Yellow seed | 70.4 (103) | 3.9 (45) | 6.1 (45) |

*DM = dry matter

than its black-seeded counterpart. Oil and protein content are negatively correlated to each other (Grami et al. 1977, Bengtsson 1985). This indicates that oil or protein or both oil and protein content in yellow-seeded B. napus can be increased to a level higher than black-seeded types through further breeding. Fibre content in yellow seed as well as in oil-free meal from yellow seed was reduced by about 55% compared to black seed or oil-free meal from black seed.

Discussion

Source of the Transparent Seed Coat Gene in the CC Genome of Yellow-Seeded B. napus Derived from the No. 06×No. 01 Cross

The seed coat colour of the parent B. napus line No. 01 (AACC) which was resynthesized from the black-seeded B. alboglabra (CC)×Yellow Sarson (B. campestris) (AA) cross is completely black. This line carries the transparent seed coat gene in its AA genome and lacks the transparent seed coat gene in its CC genome. Thus, the generation of a completely yellow-seeded B. napus from a No. 06×No. 01 cross is not possible without a transparent seed coat gene in the CC genome of the No. 06 line.

The transparent seed coat gene in the CC genome of the B. napus line No. 06 is the transparent seed coat gene of Yellow Sarson (B. campestris) which has been transferred through allosyndesis between the A- and the C-genome chromosomes during development of the line No. 06 from the interspecific cross.

The possibility that the transparent seed coat gene in the CC genome of the No.06 was directly derived from the B. carinata (BBCC) line was eliminated by the-demonstration that the synthesis of trigenomic hexaploids (AABBCC) from the yellow-seeded B. carinata×Yellow Sarson (B. campestris) crosses failed to produce a transparent seed coat colour, that is, yellow seeds.

Furthermore, from a separate study, the partly yellow-seeded CC-genomic species, B. alboglabra was resynthesised from the {(yellow-seeded B. carinata×black-seeded B. alboglabra)×black-seeded B. alboglabra} cross. Using this partly yellow-seeded B. alboglabra (CC) and yellow-seeded B. campestris Yellow Sarson (AA), B. napus (AACC) was resynthesised. The resynthesised B. napus was completely black-seeded. A similiar approach by Chen (Chen et al 1988; Chen and Heneen 1992) failed to produce any yellow-seeded B. napus.

These results clearly demonstrate that: (i) the parent, F₇ generation, yellowish brown-seeded B. napus line (No.06), developed from the {{B. carinata×Yellow Sarson (B.campestris))×B. napus} cross carries the gene for transparent seed coat colour of Yellow Sarson (B. campestris) in its CC genome and (ii) the transparent seed coat gene of B. carinata in combination with the transparent seed coat gene of Yellow Sarson (B. campestris) fails to produce yellow-seeded B. napus.

Summation

The present invention therefore provides transformed plants comprising a transparent seed coat gene.

In a preferred aspect the present invention describes the development of stable and consistent transparent-seed coat coloured i.e. yellow-seeded oilseed rape B. napus from interspecific crosses. In this respect, the transparent seed coat gene of the AA genome of Yellow Sarson (B. campestris) were transferred into the CC genome of B napus through allosyndesis between the A- and the C-genome chromosomes. The resultant B napys AACC genome, carrying the transparent seed coat gene of Yellow Sarson (B. campestris), yielded a consistent and stable yellow seed coloured B. napus plant line.

As will be apparent—particularly from the teachings of the Examples—the application of the present invention is not technically confined to a single plant variety.

All publications and patents mentioned in the above specification are herein incorporated by reference. Various modifications and variations of the described aspects of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in plant biology and/or plant biotechnology and/or plant molecular biology and/or related fields are intended to be within the scope of the following claims.

TABLE 1
Selection for yellow-seeded B. napus from interspecific cross derived trigenomic
haploids (ABC) × B. napus (AACC) cross

| | | | | | Percent | Number of plants for seed | | | |
|---|---|---|---|---|---|---|---|---|---|
| | | | | Number | plants | colour | | | |
| | Mother Plant | | Examined | plants | producing | | | Light | Yellowish |
| Parentage | Generation | Seed colour | Generation | grown | seeds | Black | Brown | brown | brown |
| (Cari × Cam) × Nap | F₁ | Black | F₂ | 272 | 39.0 | 97 | 9 | — | — |
| (Cari × Cam) × Nap | F₂ | Brown | F₃ | 116 | 85.3 | 82 | 15 | 2 | — |
| (Cari × Cam) × Nap | F₃ | Light brown | F₄ | 50 | 82.0 | — | 7 | 28 | 6 |
| (Cari × Cam) × Nap | F₄ | Yellowish | F₅ | 20 | 70.0 | — | — | 4 | 10 |
| | | brown | | | | | | | |
| (Cari × Cam) × Nap | F₅ | Yellowish | F₆ | 40 | 92.5 | — | 10 | 12 | 15 |
| | | brown | | | | | | | |
| (Cari × Cam) × Nap | F₆ | Yellowish | F₇ | 20 | 100 | — | — | 7 | 13 |
| | | brown | | | | | | | |

Cari = B. carinata (Yellow Seed), 381078,
Cam = B. campestris Yellow Sarson (Yellow Seed), 3-0166.001,
Nap = B. napus (Black Seed), 1-9007

TABLE 2
Crossability between interspecific cross derived
resynthesized B. napus lines No. 06 and No. 01 and
natural B. napus cv. Jaguar.

| | | Number seeds |
|---|---|---|
| | Number | per |
| Cross | pollination | pollination |
| No. 06 × No. 01 | 17 | 9.4 |
| No. 01 × No. 06 | 10 | 10.3 |
| No. 06 × Jaguar | 28 | 16.9 |
| Jaguar × No. 06 | 10 | 26.1 |
| No. 01 × Jaguar | 15 | 9.8 |
| Jaguar × No. 01 | 31 | 18.5 |

TABLE 3
Selection for yellow seed in the progenies generated from
crossing two resynthesized B. napus lines, No. 06 × No. 01.

| | | | | | Black/ | | Partly | | Yellow | | | |
|---|---|---|---|---|---|---|---|---|---|---|---|---|
| | | Examined | Number | Total | brown | | yellow | | brown | | Yellow | |
| Mother plant | | plant | families | number | seed | | seed | | seed | | seed | |
| Generation | Seed colour | generation | grown | plants | No. | % | No. | % | No. | % | No. | % |
| F1 | — | F2 | — | 259 | 199 | 76.8 | 51 | 19.7 | 9 | 3.5 | — | — |
| F2 | Yellow | F3 | 9 | 217 | 38 | 17.5 | 98 | 45.2 | 80 | 36.9 | 1 | 0.5 |
| | brown | | | | | | | | | | | |
| F3 | Yellow | F4 | 1 | 22 | — | — | 1 | 4.5 | 12 | 54.5 | 9 | 40.9 |
| | Yellow | F4 | 8 | 103 | 17 | 16.5 | 63 | 61.2 | 23 | 22.3 | — | — |
| | brown | | | | | | | | | | | |
| F4 | Yellow | F5 | 4 | 55 | — | — | — | — | 12 | 21.8 | 43 | 78.2 |
| F5 | Yellow | F6 | 14 | 122 | — | — | — | — | 5 | 4.1 | 117 | 95.9 |
| F6 | Yellow | F7 | 2 | 25 | — | — | — | — | — | — | 25 | 100.0 |

TABLE 4
Crossability of yellow seeded B. napus as
female with natural B. napus.

| Generation | | | |
|---|---|---|---|
| of | | | |
| yellow | Number | | |
| seeded | yellow | | Number |
| family | seeded | Number | seeds per |
| crossed | family used | pollinations | pollination |
| F4 | 1 | 30 | 3.0 |
| F6 | 6 | 208 | 4.4 |
| F7 | 2 | 55 | 4.8 |
| F8 | 2 | 70 | 6.8 |
| F9 | 3 | 55 | 6.9 |

TABLE 5
Segregation for seed colour in doubled haploid (DH) lines from cross between
yellow-seeded B. napus (13-217 and 13-219) and black-seeded natural B. napus (Polo and Dakini).

| | | Black/ | | | | | | | | |
|---|---|---|---|---|---|---|---|---|---|---|
| | Total | dark | Reddish | Partly | Yellow | | Segregation | | Segregation | |
| | DH | brown | brown | yellow | brown | Yellow | 15:1^{a} | | 7:1^{b} | |
| Cross | lines | seed | seed | seed | seed | seed | X² | p | X² | p |
| Polo × 13-217 | 75 | 52 | 8 | 8 | 3 | 4 | 0.008 | 0.9-0.95 | 0.43 | 0.5-0.7 |
| Dakini × 13-217 | 70 | 43 | 13 | 7 | 4 | 3 | 0.19 | 0.5-0.7 | 0.20 | 0.5-0.7 |
| Polo × 13-219 | 75 | 52 | 5 | 7 | 4 | 7 | 0.75 | 0.3-0.5 | 0.15 | 0.5-0.7 |
| Dakini × 13-219 | 67 | 48 | 8 | 5 | 3 | 3 | 0.12 | 0.7-0.9 | 0.48 | 0.3-0.5 |
| Total | 287 | 195 | 34 | 27 | 14 | 17 | 0.01 | 0.9-0.95 | 0.61 | 0.3-0.5 |

^{a}Segregation tested after pooling data of black/dark brown, reddish brown, partly yellow and yellow-brown seed into one class and yellow seed into the other class
^{b}Segregation tested after pooling data of black/dark brown, reddish brown and partly yellow seed into one class, and yellow-brown and yellow seed into the other class

TABLE 6
Segregation for petal colour and erucic acid (C_{22:1})
content in doubled haploid (DH) lines from cross between
yellow-seeded B. napus (13-217 and 13-219) and natural
B. napus (Polo and Dakini).

| | |
|---|---|
| Yellow petal | White petal |

| | − C_{22:1} | + C_{22:1} | − C_{22:1} | + C_{22:1} |
|---|---|---|---|---|
| Polo × 13-217 | 3 | — | — | 7 |
| Dakini × 13-217 | 10 | — | 1 | 16 |
| Polo × 13-219 | 8 | — | 1 | 3 |
| Dakini × 13-219 | 4 | 1 | — | 7 |
| Total | 25 | 1 | 2 | 33 |

The absence (3.1% or less) and presence (15.8-33.4%) of erucic acid content is indicated by (−) and (+) signs respectively.

TABLE 7
Segregation for glucosinolate(s) (GLS) (μmol/g seed)
content in doubled haploid lines from cross between yellow-
seeded B. napus (13-217 and 13-219) and natural
B. napus (Polo and Dakini).

| | <20 μmol | >20 μmol | Segregation, 1:15 | |
|---|---|---|---|---|
| Cross | GLS | GLS | X² | p |
| Polo × 13-217 | 3 | 51 | 0.005 | 0.9-0.95 |
| Dakini × 13-217 | 2 | 55 | 0.34 | 0.5-0.7 |
| Polo × 13-219 | 1 | 44 | 0.65 | 0.3-0.5 |
| Dakini × 13-219 | 1 | 45 | 0.70 | 0.3-0.5 |
| Total | 7 | 195 | 2.22 | 0.1-0.2 |

References

Anjou K, Lönnerdal B, Uppström B, Åman P. 1977. Composition of seeds from some Brassica cultivars. Swedish J. Agric. Res. 7:169-178.

Association of Official Analytical Chemists, 1990. Official methods of analysis of the Association of Official Analytical Chemists (ed. K. Helrich), 15^{th} edition, Volume 2, Published by Association of Official Analytical Chemists, Virginia, USA.

Attia T, Busso C, Röbbelen G. 1987. Digenomic triploids for an assessment of chromosome relationships in the cultivated diploid Brassica species. Genome 29:326-330.

Attia T, Röbbelen G. 1986. Cytogenetic relationship within cultivated Brassica analyzed in amphihaploids from the three diploid ancestors. Can J Genet Cytol 28:323-329.

Barcikowska B, Zwierzykowska E, Balicka M. 1987. On the way to yellow-seeded Brassica napus L.—Hybrids of B. campestris×B. oleracea and B. oleracea×B. carinata yellow seeded. Proc. 7th Int. Rapeseed Congr., Poland 2, 492-495.

Bell J M, Shires A. 1982. Composition and digestibility by pigs of hull fractions from rapeseed cultivars with yellow or brown seed coats. Can. J. Anim. Sci. 62:557-565.

Bengtsson, L., 1985: Improvement of rapeseed meal quality through breeding for high protein content. A Ph.D. Thesis. The Swedish University of Agricultural Sciences, Department of Crop Genetics and Breeding, Svalöv, Sweden.

Busso C, Attia T, Röbbelen G. 1987. Trigenomic combinations for the analysis of meiotic control in the cultivated Brassica species. Genome 29:331-333.

Chen B Y, Heneen W K. 1992. Inheritance of seed colour in Brassica campestris L. and breeding for yellow-seeded B. napus L. Euphytica 59:157-163.

Chen B Y, Heneen W K, Jonsson R. 1988. Resynthesis of Brassica napus L. through interspecific hybridization between B. alboglabra Bailey and B. campestris L. with special emphasis on seed colour. Plant Breed. 101:52-59.

Daun J K, DeClercq D R. 1988. Quality of yellow and dark seeds in Brassica campestris canola varieties Candle and Tobin. JAOCS 65:122-126.

Galbraith D W, Harkins K R, Maddox J M, Ayres N M, Sharma D P, Firoozabady E. 1983. Rapid flow cytometric analysis of the cell cycle in intact plant tissues. Science 220:1049-1051.

Gland A. 1981. Doubling chromosomes in interspecific hybrids by colchicine treatment. Eucarpia Cruciferae Newsletter 6:20-22.

Grami, B., R. J. Baker, and B. R. Stefansson, 1977: Genetics of protein and oil content in summer rape: heritability, number of effective factors, and correlations. Can. J. Plant Sci. 57, 937-943.

Hu X-J. 1988. Studies on the relationship between seedcoat colour and pigment content in different types of rapeseed cultivars. Cruciferae Newsletter 13:44-45.

Leung J, Fenton T W, Mueller M M, Clandinin D R. 1979. Condens tannins of rapeseed meal. J. Food Sci. 44:1313-1316.

Lichter R. 1989. Efficient yields of embryoids by culture of isolated microspores of different Brassicaceae species. Plant Breed 103:119-123.

Liu H-L. 1983. Studies on the breeding of yellow seeded Brassica napus. Proc. 6th Int. Rapeseed Conf., Paris. pp. 637-641.

Liu H-L, Gao Y-T. 1987. Some fundamental problems conducted from the studies on the breeding of yellow-seeded Brassica napus L. Proc. 7th Int. Rapeseed Congr., Poznan, Poland. pp. 476-480.

Murashige I, Skoog, F. 1962. A revised medium for rapid growth and bioassays with tobacco tissue cultures. Physiol Plant 15:473-497.

Murphy R W, Sites Jr. J W, Buth D G, Haufler C H. 1990. Isozyme electrophoresis. In: Hillis D M and Moritz C (eds): Molecular systematics, pp. 45-126. Sinaur Associates, Sunderland, USA.

Rashid A, Rakow G, Downey R K. 1994. Development of yellow seeded Brassica napus through interspecific crosses. Plant Breed. 112:127-134.

Sabharwal P S, Dolezel J. 1993. Interspecific hybridization in Brassica: Application of flow cytometry for analysis of ploidy and genome composition in hybrid plants. Biologia Plantarum 35:169-177.

Schwetka A. 1982. Inheritance of seed colour in turnip rape (Brassica campestris L.). Theor. Appl. Genet. 62:161-169.

Shirzadegan M, Röbbelen G. 1985. Influence of seed colour and hull proportion on quality properties of seeds in Brassica napus L. Fette Seifen Anstrichmittel 87:235-237.

Simbaya, J., Slominski, B A, Rakow, G, Campbell, L D, Downey, R K, Bell J M 1995. Quality characteristics of yellow-seeded Brassica seed meals: Protein, carbohydrates and dietary fiber components. J. Agric. Food Chem. 43: 2062-2066.

Slominski B A, Campbell L D, Guenter W. 1994. Carbohydrates and dietary fiber components of yellow-seeded and brown-seeded canola. J. Agric. Food Chem. 42:704-707.

Stringam, G. R., D. I. McGregor, and S. H. Pawlowski, 1974: Chemical and morphological characteristics associated with seedcoat color in rapeseed. Proc. 4th Int. Rapeseed Conf., Giessen, Germany, 99-108.

Smith D B, Parsons D G, Starr C. 1985. A simple and rapid method of quantitatively measuring the glucosinolate(s) concentration of rapeseed. J. agric Sci., Camb. 105:597-603.

Stringam G R 1980. Inheritance of seed color in turnip rape. Can. J. Plant Sci., 60, 331-335.

Stringam G R, McGregor D I, Pawlowski S H. 1974. Chemical and morphological characteristics associated with seedcoat color in rapeseed. Proc. 4th Int. Rapeseed Conf., Giessen, Germany. pp.99-108.

Tang Z L, Li J N, Zhang X K, Chen L, Wang R. 1997. Genetic variation of yellow-seeded rapeseed lines (Brassica napus L.) from different genetic sources. Plant Breed 116:471-474.

Theander O, Åman P, Miksche G E, Yasuda S. 1977. Carbohydrates, polyphenol, and lignin in seed hulls of different colors from turnip rapeseed. J. Agric. Food Chem. 25:270-273.

Van Deynze A E, Beversdorf W D, Pauls K P. 1993. Temperature effects on seed color in black- and yellow-seeded rapeseed. Can. J. Plant Sci. 73:383-387.

Van Deynze A, Pauls K P. 1994. The inheritance of seed colour and vernalization requirement in Brassica napus using doubled haploid populations. Euphytica 74:77-83.

Vaughan J G. 1970. Cruciferae. In: J G Vaugan (ed) The structure and utilization of oilseeds. Chapman and Hall, London, UK. pp. 49-62.

Woods D L. 1980. The association of yellow seed coat with other characteristics in mustard B. juncea. Cruciferae Newsletter 5:23-24.

Zaman M W. 1988. Limitations for introgression of yellow seed coat colour in Brassica napus. Sveriges Utsadesförenings Tidskrift, 98:157-161.

1. A transformed CC genome comprising an exogenous transparent seed coat gene obtained from an AA genome.

2. A transformed CC genome according to claim I wherein the AA genome is an AA genome obtained from any one of Brassica campestris, Brassica napus and Brassica juncea, preferably from Brassica campestris.

3. A transformed CC genome according to claim 1 or claim 2 wherein the transformed CC genome is a transformed Brassica napus CC genome.

4. A transformed plant, plant cell or plant tissue comprising an exogenous transparent seed coat gene.

5. A transformed plant, plant cell or plant tissue comprising an exogenous transparent seed coat gene obtained from an AA genome.

6. A transformed plant, plant cell or plant tissue according to claim 5 wherein the AA genome is obtained from any one of Brassica campestris, Brassica napus and Brassica juncea, preferably from Brassica campestris.

7. A transformed plant, plant cell or plant tissue according to any one of claims 4-6 wherein the transformed plant, plant cell or plant tissue is a transformed Brassica plant, plant cell or plant tissue.

8. A transformed plant, plant cell or plant tissue according to claim 7 wherein the transformed plant, plant cell or plant tissue is a transformed Brassica napus plant, plant cell or plant tissue.

9. A transformed plant, plant cell or plant tissue according to any one of claims 4-8 wherein the transformed plant, plant cell or plant tissue is capable of yielding seeds with a transparent seed coat or is capable of yielding plants having seeds with a transparent seed coat.

10. A transformed plant according to any one of claims 4-9 wherein the transformed plant is non-sterile.

11. A yellow seed comprising a transparent seed coat gene as defined in claim 1 or claim 2 and preferably any one combination of:
(i) zero erucic acid or a low level of erucic acid or a medium level of erucic acid or a high level of erucic acid; and
(ii) zero glucosinolate(s) or a low level of glucosinolate(s) or a medium level of glucosinolate(s) or a high level of glucosinolate(s).

12. A yellow seed according to claim 11 wherein the seed is a transformed Brassica napus yellow seed having a low level of erucic fatty acid and a low level of glucosinolate(s).

13. A yellow seed according to claim 11 wherein the yellow seed is a transformed Brassica napus yellow seed having a medium level of erucic fatty acid and a high level of glucosinolate(s).

14. A yellow seed according to any one of claims 11-13 wherein the seed has an increased level of seed oil and protein compared to the seed oil and protein level in a black seed or a brown seed.

15. A yellow seed according to any one of claims 11-14 wherein the yellow seed has an oil and protein content of at least about 70% seed dry matter.

16. A yellow seed according to any one of claims 11-14 wherein the seed has a decreased level of seed fibre compared to the seed fibre level in a black seed or a brown seed.

17. A yellow seed according to claim 16 wherein the seed has a fibre content of not more than about 8% oil free meal.

18. Use of a yellow seed according to any one of claims 11-17 to prepare a seed oil or a seed meal.

19. A seed oil or a seed meal produced from the yellow seeds according to any one of claims 11-17.

20. A method for increasing the levels of seed oil and protein and reducing the levels of fibre in a seed wherein the method comprises: transferring the transparent seed coat gene of an AA genome of a first Brassica plant, plant tissue or plant cell into a CC genome of a second Brassica plant, plant tissue or plant cell.

21. A method according to claim 20 wherein the AA genome is obtained from any one of Brassica campestris, Brassica napus and Brassica juncea, preferably from Brassica campestris.

22. A method according to claim 20 or claim 21 wherein the CC genome is a Brassica napus CC genome.

23. A method according to any one of claims 20-22 wherein the seed has an increased level of seed oil and protein compared to the seed oil and protein level in a black or a brown seed.

24. A method according to any one of claims 20-23 wherein the seed has an oil and protein content of at least about 70% seed dry matter.

25. A method according to any one of claims 20-24 wherein the seed has a decreased level of seed fibre compared to the seed fibre level in a black or a brown seed.

26. A method according to any one of claims 20-25 wherein the seed has a fibre content of not more than about 8% oil free meal.

27. A transformed Brassica napus plant capable of yielding seeds with a transparent seed coat.

28. A seed oil or a seed meal comprising an oil and protein content of at least about 70% seed dry matter and a fibre content of not more than about 8% oil free meal.

29. Use of an AA genome as a vector for delivery of one or more genes of interest to a heterologous genome.

30. Use according to claim 29 wherein the AA genome is obtained from any one of Brassica campestris, Brassica napus and Brassica juncea, preferably from Brassica campestris.

31. Use according to claim 29 or claim 30 wherein the CC genome is a Brassica napus CC genome.

32. A transparent seed coat encoded by a transparent seed coat gene obtainable from. NCIMB 40991 and/or NCIMB 40992.

33. A transparent seed coat.

34. A transformed Brassica genome comprising an exogenous transparent seed coat gene.

35. A transformed Brassica napus plant wherein the Brassica napus comprises a transparent seed coat gene obtained from the AA genome of Brassica as described herein and with reference to the accompanying Figures.

36. A method for preparing a Brassica plant that produces seeds having a stable and uniform yellow phenotype comprising:
crossing a first Brassica plant, having a CC genome homozygous for a first transparent seed coat gene, said first transparent seed coat gene derived from a first AA genome of Brassica campestris, with a second Brassica plant having a second AA genome of Brassica campestris homozygous for a second transparent seed coat gene to produce an offspring Brassica plant heterozygous for said first and second transparent seed coat genes, and self-crossing the offspring Brassica plant to produce said Brassica plant homozygous for said first and second transparent seed coat genes and wherein said Brassica plant produces seeds having a stable and uniform yellow phenotype.

37. The method according to claim 36 wherein the first Brassica plant is Brassica napus.

38. The method according to claims 37 wherein the Brassica napus produces yellowish-brown seed.

39. The method according to claim 36 wherein the second Brassica plant is resynthesized Brassica napus.

40. The method accordilng to claim 39 wherein the resynthesized Brassica napus is formed by the steps comprising crossing Brassica alboglabra and Brassica campestris to form an embryo comprising an amphihaploid genome (AC), rescuing said embryo using an embryo rescue technique, and duplicating said amphihaploid genome using chromosome doubling wherein said resynthesized Brassica napus is formed.

41. The method according to claim 36 wherein the first transparent seed coat gene is transferred from said first AA genome of Brassica campestris to said CC genome through allosyndesis.

42. The method according to claim 36 wherein the first Brassica plant is formed by the steps of:
(a) crossing a multigenomic plant, itself the progeny of a cross between a first Brassica species and said Brassica campestris having said first AA genome homozygous for said first transparent seed goat gene, with natural Brassica napus having a natural AA genome and natural CC genome to form a progeny hybrid plant;
(b) transferring by allosyndesis said first transparent seed coat from said first AA genome to said natural CC genome of Brassica napus through self-crossing of said progeny hybrid plant through a plurality of generations to produce said first Brassica plant having the CC genome homozygous for the first transparent seed coat gene.

43. The method according to claim 41 wherein said first Brassica species is Brassica carinata.

44. The method according to claim 39, wherein the resynthesized Brassica napus plant is formed by the steps of:
(a) crossing a first parent Brassica plant with a second parent Brassica plant having said second AA genome of claim 36 homozygous for said second transparent seed coat gene to form a progeny embryo; and
(b) forming said resynthesized Brassica napus plant by performing embryo rescue and chromosome doubling on said progeny embryo; wherein said resynthesized Brassica napus plant comprises said second AA genome homozygous for said second transparent seed coat gene.

45. The method according to claim 44 wherein said first parent Brassica plant is Brassica alboglabra having a CC genome and black seeds.

46. The method according to claim 44 wherein said second parent Brassica plant is Brassica campestris having an AA genome homozygous for transparent seed coat gene.

47. A method for preparing a Brassica plant that produces seeds having a stable and uniform yellow phenotype comprising transferring an AA genome homozygous for a first transparent seed coat gene from Brassica campestris and CC genome homozygous for a second transparent seed coat gene from Brassica campestris to Brassica napus.

48. A method for preparing a Brassica plant that produces seeds having a stable and uniform yellow phenotype comprising the step of transferring a first transparent seed coat gene from a first AA genome of Brassica campestris to the CC genome of Brassica napus.

49. The method according to claim 48 wherein said Brassica plant is Brassica napul having said CC genome comprising said first transparent seed coat gene from said first AA genome of Brassica campestris and a second AA genome homozygous for a second transparent seed coat gene from Brassica campestris.

50. The method according to claim 48 wherein the step of transferring occurs by a biotechnological technique.

51. The method according to claim 48 wherein the step of transferring occurs by a selective cross-breeding procedure.

52. The method according to claim 51 wherein the step of transferring by a selective cross-breeding procedure includes the step of allosyndesis.

53. The method according to claim 48 wherein said Brassica plant is Brassica napus 13-217, deposit number NCIMB 40991.

54. The method according to claim 48 wherein said Brassica plant is Brassica napus 13-291, deposit number NCIMB 40992.
